(19) United States
(12) Patent Application Publication
(10) Pub. No.: US 2024/0016671 A1
(43) Pub. Date: Jan. 18, 2024

(54) Skin Patch For Nasal Drainage

(71) Applicant: ASO LLC, Sarasota, FL (US)

(72) Inventors: Brooke A. Hagerty, Bradenton, FL (US); Edmund A. Sinda, Bradenton, FL (US)

(21) Appl. No.: 18/221,938

(22) Filed: Jul. 14, 2023

Related U.S. Application Data
(60) Provisional application No. 63/389,466, filed on Jul. 15, 2022.

(51) Int. Cl. A61F 13/84; A61F 13/58; A61F 13/15

(52) U.S. Cl. CPC A61F 13/8405; A61F 13/58; A61F 2013/15008; A61F 2013/8435

(57) ABSTRACT

A skin patch for delivering scent to a human nose includes a flexible base member configured to be adhered between a base of the nose and a vermillion border. The flexible base member defines an outer perimeter having a left edge, a right edge, an upper edge extending from the left edge to the right edge, and a lower edge opposite the upper edge and extending from the left edge to the right edge. The flexible base member includes a first side and a second side opposite the first side, the second side including a skin contact adhesive. A plurality of perforations through the flexible base member extend from the first side to the second side. The perforations are distributed across the flexible base member. A non-perforated section of the flexible base member carries a scented portion.

### CROSS-REFERENCE TO RELATED APPLICATION

This claims the benefit of priority from Application No. 63/389,466, filed Jul. 15, 2022, which is incorporated by reference in its entirety.

### FIELD

This relates to the field of treating nasal congestion and/or protecting skin from nasal drainage, more particularly, to using a skin patch worn on the face.

### BACKGROUND

Nasal congestion and drainage is a common symptom of upper respiratory problems such as allergies, colds, and influenza. Because young children find it difficult to maintain a clean, dry face during times of nasal drainage, they often experience discomfort due to skin irritation from the mucus itself and from wiping away the mucus.

Many people treat nasal congestion by applying a substance such as menthol or eucalyptus around the nose to assist in clearing the nasal congestion. Although such substances are therapeutically effective, the wearer often finds them messy and wipes them off when they blow their nose.

### BRIEF SUMMARY

What is needed is a skin patch that can be worn on the face for protecting the skin from nasal drainage and/or treating nasal congestion. The skin patch and related methods described here fulfill this need. The skin patch may be particularly useful on children.

A skin patch for delivering scent to a human nose includes a flexible base member configured to be adhered between a base of the nose and a vermillion border. The flexible base member defines an outer perimeter having a left edge, a right edge, an upper edge extending from the left edge to the right edge, and a lower edge opposite the upper edge and extending from the left edge to the right edge. The flexible base member includes a first side and a second side opposite the first side, the second side including a skin contact adhesive. A plurality of perforations through the flexible base member extend from the first side to the second side. The perforations are distributed across the flexible base member. A non-perforated section of the flexible base member carries a scented portion.

The skin patch may also include one or more of the following features.

The scented portion may include menthol, camphor, and/or eucalyptus.

The non-perforated section may be disc-shaped and is positioned at a center of the flexible base member.

The perforations may be circular.

The perforations may be evenly distributed across the flexible base member.

The scented portion may include a liquid absorbent material.

The outer perimeter may define a trapezoid with the upper edge being longer than the lower edge.

The flexible base member may carry the scented portion on the first side.

The flexible base member may carry the scented portion within the flexible base member.

The scented potion may remain on the flexible base member when nasal drainage is wiped from the flexible base member.

An example of a method includes delivering scent to a human nose by adhering a skin patch to an upper lip. The skin patch has flexible base member that defines an outer perimeter having a left edge, a right edge, an upper edge extending from the left edge to the right edge, and a lower edge opposite the upper edge and extending from the left edge to the right edge. The flexible base member also includes a first side and a second side opposite the first side, the second side including a skin contact adhesive. A plurality of perforations through the flexible base member extend from the first side to the second side. The perforations are distributed across the flexible base member. A non-perforated section of the flexible base member carries a scented portion.

The skin patch may also include one or more of the following features.

The scented portion may include menthol, camphor, and/or eucalyptus.

The non-perforated section may be disc-shaped and positioned at a center of the flexible base member.

The perforations may be circular.

The perforations may be evenly distributed across the flexible base member.

The scented portion may include a liquid absorbent material.

The outer perimeter may define a trapezoid with the upper edge being longer than the lower edge.

The flexible base member may carry the scented portion on the first side.

The flexible base member may carry the scented portion within the flexible base member.

The scented potion may remain on the flexible base member when nasal drainage is wiped from the flexible base member.

### BRIEF DESCRIPTION OF THE DRAWINGS

FIG. 1 is a diagram of a human face.

FIG. 2 is a diagram of a human face wearing a skin patch.

FIG. 3 is a front view of a first example of the skin patch.

FIG. 4 is a back view thereof.

FIG. 5 is a side view thereof.

FIG. 6 is a front view of a second example of the skin patch.

FIG. 7 is a back view thereof.

FIG. 8 is a side view thereof.

FIG. 9 is a front view of a third example of the skin patch.

FIG. 10 is a back view thereof.

FIG. 11 is a side view thereof.

FIG. 12 is a front view of several additional examples of the skin patch.

FIG. 13 is a front view of yet another example of the skin patch.

### DETAILED DESCRIPTION OF EXAMPLE EMBODIMENTS

This disclosure describes examples, but not all possible examples of the skin patch and methods. Where a particular feature is disclosed in the context of a particular example, that feature can also be used, to the extent possible, in combination with and/or in the context of other examples. The skin patch and methods may be embodied in many different forms and should not be construed as limited to only the features and examples described here.

To provide a reference frame for the description of the skin patch, certain features of a human face 100 are first described by referring to FIG. 1. The human face 100 includes a nose 102 extending from a root 104 to a tip 106. A base 108 defines the bottom of the nose 102 and includes a pair of nares 110. The nares 110 are laterally defined by ala nasi 112. Extending from the base 108 to a vermillion border 114 is an upper lip 116, which includes a philtrum 118.

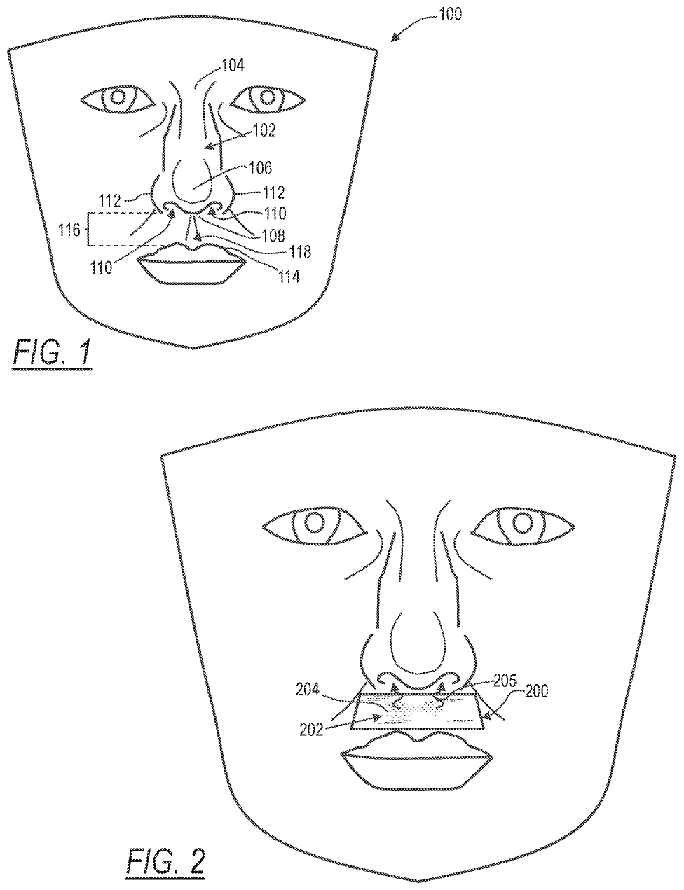

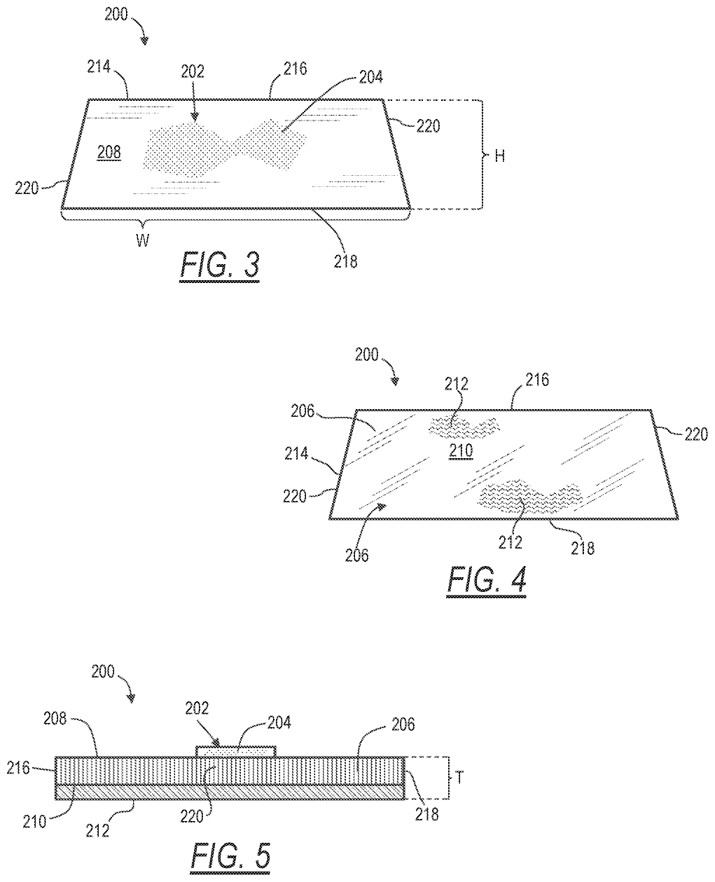

Referring to FIG. 2, a first example of the skin patch 200 is designed to be positioned on the upper lip 116 between the vermillion border 114 and base 108. The skin patch 200 includes a scented portion 202 which is positioned beneath the nares 110 for delivering a scent formula 204 with a scent 205 directly into the nares 110. The scent formula 204 may be selected to provide a therapeutic benefit when the wearer is experiencing nasal congestion. FIGS. 3-5 show additional details of this skin patch 200.

The skin patch 200 may provide one or more of the following advantages.

Some examples of the skin patch 200 provide a physical barrier between the skin of the upper lip 116 and mucus from nasal drainage by covering and protecting the skin of the upper lip 116 during instances of nasal drainage. This can prevent or mitigate skin irritation due to the mucus itself or from wiping away the mucus.

The skin patch 200 provides a physical barrier between the scent formula 204 and the skin of the upper lip 116 because the skin patch 200 carries the scent formula 204 thereon. This substantially prevents the scent formula 204 from contacting the upper lip 116 and causing discomfort or skin irritation due to sensitivity of the wearer to the scent formula 204.

In some examples of the skin patch 200, the scent formula 204 may be relatively permanent on the skin patch 200, which means the scent formula 204 can remain in place when contacted by nasal drainage or when nasal drainage is wiped away.

In some examples of the skin patch 200, the scent formula 204 may be absent. In such examples, the skin patch 200 functions to protect the skin without providing a scent.

In some examples of the skin patch 200, the skin patch 200 is removable and reusable.

In some examples, the skin patch 200 is attached to the upper lip with an adhesive that permits gentle removal so as to avoid or minimize any damage to the fragile skin of the upper lip 116 and philtrum 118.

In some examples, the skin patch 200 is designed in such a way that it does not obstruct the nares 110 such that it does not contribute to breathing difficulty when the wearer experiences nasal congestion.

In some examples, the skin patch 200 is designed in such a way that it does not impede speech or mouth functions due to the flexibility of the skin patch and having its edges remain outside the vermillion border 114.

The first example of the skin patch 200 shown in FIG. 2 will now be described in more detail by referring to FIGS. 3-5. The skin patch 200 includes a base member 206 having a first surface 208 and a second surface 210 with an adhesive 212 on the second surface 210. On the first surface 208 is a scented portion 202 carrying a scent formula 204.

The base member 206 includes an outer perimeter 214 defined by an upper edge 216, lower edge 218 opposite the upper edge 216, and a pair of opposed lateral edges 220. The outer perimeter 214 is designed to fit on the upper lip 116 beneath the base 108 of the nose 102 and vermillion border 114 and over the philtrum 118. The vertical height H of the base member 206 between the upper edge 216 and lower edge 218 is less than a distance between the base 108 and vermillion border 114. A width W of the base member 206 between the lateral edges 220 will typically approximate the lateral width of the wearer's lips. Among different examples of the skin patch, the height H and width W can vary to meet desired specifications, fit on different sized faces, and accommodate different aesthetic designs.

In the example of FIGS. 2-5, the outer perimeter 214 defines a trapezoidal shape with the upper edge 216 being shorter than the lower edge 218. This trapezoidal shape is advantageous because it provides a taper from the base 108 of the nose 102 to the vermillion border 118. Having a shorter upper edge 216 near the base 108 of the nose 102 aids with comfort.

The base member 206 may be made of thin, flexible material. In certain examples, the base member 206 and adhesive 212 combined have a thickness T of 5 mm to 0.1 mm, 4 mm to 0.1 mm, 3 mm to 0.1 mm, 2 mm to 0.1 mm, or 1 mm to 0.1 mm. This small thickness is advantageous because it allows the skin patch 200 to lay almost flush with the wearer's skin when worn so that the skin patch 200 does not substantially block air flow into the nares 110.

In some examples, the material from which the base member 206 is made is a plastic or plastic-like material such as polyethylene, polyurethane, silicone, or the like. This material may, if desired, be breathable and have a WVTR of greater than 0 g/m² per 24 hours and up to 10,000 g/m² per 24 hours.

In some examples, the material from which the base member 206 is made is a fabric or fabric-like material. The fabric or fabric-like material may, if desired, be capable of absorbing the scent formula 204.

In some examples, the material from which the base member 206 is made is a foam or foam-like material. The foam or foam-like material may, if desired, be capable of absorbing the scent formula 204.

In some examples, the first surface 206 includes a decorative texture or design that mimics the appearance of a mustache.

The adhesive 212 is a thin layer of adhesive material applied to the second surface 210 of the base member 206. The adhesive material is selected from skin contact adhesives that can adhere firmly to the skin, but are removable without damaging the skin. Examples of skin contact adhesives include low trauma adhesives such as acrylic adhesives, silicone adhesives, or the like. Other examples include a hydrogel, hydrocolloid, or the like. The adhesive may include one or more additives that provides a benefit to the wearer's skin such as, for example, vitamins, vitamin E, zinc oxide, or the like.

The scent formula 204 is a chemical composition that includes a scent compound. The scent compound may be therapeutically effective for mitigating nasal congestion and/or coughing. Examples of the scent formula may include the scent compound in a suitable liquid, liquid-like, wax, or wax-like carrier. Such carriers may include water, oil, oil-based, wax, or wax-based materials in which the scent compound is dissolved and may have a low viscosity similar to water or a high viscosity similar to a gel or waxy substance. Certain examples of the scent formula 204 include essential oils and scented petrolatum products. Certain examples of the scent formula 204 may include menthol, peppermint oil, eucalyptus, camphor, lavender, cinnamon, pine, any aromatherapy scent, or the like.

In the first example, the scented portion 202 with the scent formula 204 is on the first surface 208 of the base member 206. In one such scenario, the scent formula 204 may be obtained from a scent formula container and an amount of the scent formula 204 is applied to the first surface 208 using an applicator such as a cotton swab, a finger, a dropper, a brush, or the like, which can be provided to a user in a kit with multiple applicator and skin patch 200.

In another scenario, the scented portion 202 is attached to the first surface 208 and is a liquid absorbent material with the scent formula 204 distributed within the liquid absorbent material. The liquid absorbent material is capable of absorbing the scent formula 204 and holding the scent formula 204 therein. Examples of liquid absorbent materials include polyester, lyocell, wool, cotton, nylon, hydrocolloid, hydrogel, bamboo, microfiber, lycra, polyurethane, foam, or the like. The liquid absorbent material may also be capable of absorbing mucus discharged from the wearer's nose. The liquid absorbent material may also be capable of maintaining the scent formula 204 therein when mucus contacts the skin patch or when the wearer blows or wipes the wearer's nose.

In yet another scenario, the scent formula 204 is distributed within a gel that is attached to the first surface 208. The gel is capable of containing the scent formula 204 and holding the scent formula 204 therein. Examples of gel materials include hydrocolloids, hydrogels, or the like.

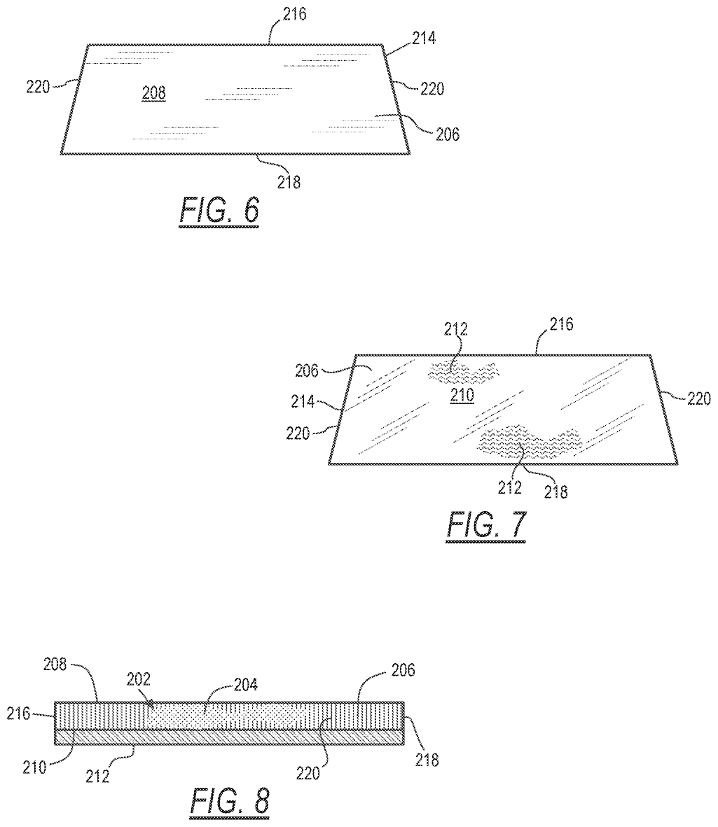

A second example of the skin patch 200 will now be described by referring to FIGS. 6-8. In this second example, and any other subsequent example of the skin patch 200 described herein, the same reference numerals are used to refer to corresponding features and the above description of the first example also applies.

Referring to FIGS. 6-8, for the second example of the skin patch 200, the scent formula 204 is distributed within the base member 206 material rather than being applied on the first surface 208. In this example, the material from which the base member 206 is made may be fabric or fabric-like material and/or a foam or foam-like material. The fabric, fabric-like, foam, or foam-like material is capable of absorbing the scent formula 204 so that the scent formula is within the base member 206.

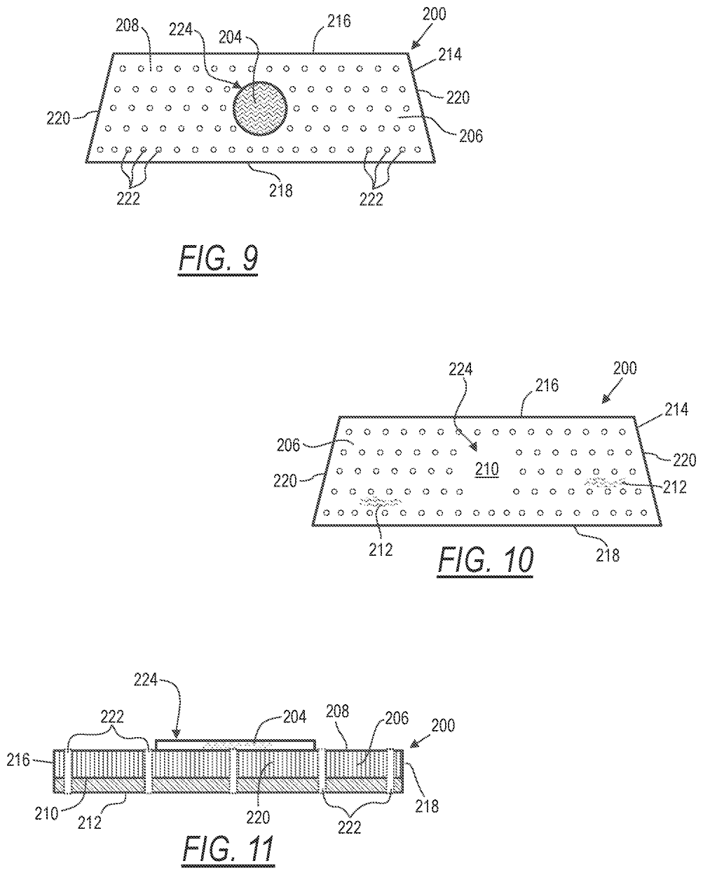

A third example of the skin patch 200 will now be described by referring to FIGS. 9-11. In this third example of the skin patch 200, the first surface 208, second surface 210, and adhesive 212 define a plurality of perforations 222 that extend through the base member 206 and adhesive 212. These perforations are distributed across the skin patch 200 to provide additional comfort to the wearer by allowing water vapor to pass through and make the skin patch 200 more flexible.

The third example of the skin patch 200, also includes a non-perforated section 224 of the first surface 208 that carries the scent formula 204. The scent formula 204 may be obtained from a scent formula container and an amount of the scent formula 204 is applied to the first surface 208 using an applicator. Alternatively, the scent formula 204 is distributed within a liquid absorbent material that is attached to the first surface 208 at the non-perforated section 224. The scent formula 204 may otherwise be distributed within a gel that is attached to the first surface 208 at the non-perforated section 224.

Although the non-perforated section 224 is disk-shaped in the example shown, it could have many other different shapes in other examples.

Referring to FIG. 12, other examples of designs of the skin patch 200 are shown. These designs include several mustache-shaped skin patch 200 that may be amusing for children to wear.

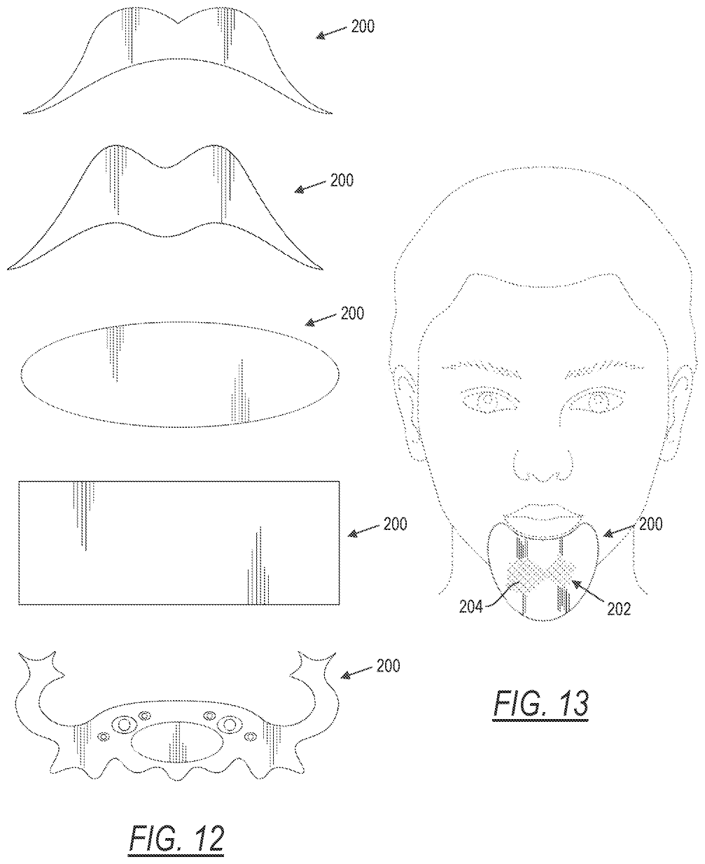

Referring to FIG. 13, yet another example of the skin patch 200 is worn on the chin. This example may emulate a beard or may be used when the wearer would prefer not to wear the skin patch on the upper lip. If desired, however, the wearer may wear a skin patch 200 on the upper lip and another skin patch 200 on the chin as desired.

It should be understood that any feature disclosed in the context of a particular example of the skin patch 200, may be used on any of the other examples skin patch 200 even though such a feature might not be shown in the drawings of a particular example or described in connection with a particular example.

The skin patch 200 may be used in a method of covering facial skin and/or delivering a scent to the nose. The method includes applying the skin patch 200 to the wearer's upper lip 116 or chin by pressing the adhesive 212 against the skin. The skin patch is preferably positioned so that the outer perimeter 214 does not contact the base 108 of the nose 102 or the vermillion border 114. This positioning minimizes any negative impact of the skin patch interfering with lip movement or flow of air through the nose 102.

If the skin patch 200 includes a scent formula 204, when the wearer inhales, scent from the scent formula 204 is entrained in the inhaled air, which delivers the scent through the nares 110 into the nose 102. The scent may symptoms of nasal congestion, such as runny nose, nasal inflammation, and/or coughing. As used herein, the term “mitigate” refers to lessening the wearer's perception of severity of the symptoms.

The skin patch and related methods are not limited to the details and features described in connection with the examples described here. There are numerous variations and modification of the skin patch and methods that may be made without departing from the scope of what is claimed.

That which is claimed is:

1. A skin patch for delivering scent to a human nose, the skin patch comprising:
a flexible base member configured to be adhered between a base of the nose and a vermillion border; the flexible base member defining an outer perimeter having a left edge, a right edge, an upper edge extending from the left edge to the right edge, and a lower edge opposite the upper edge and extending from the left edge to the right edge; the flexible base member including first side and a second side opposite the first side, the second side including a skin contact adhesive;
a plurality of perforations through the flexible base member extending from the first side to the second side, the perforations being distributed across the flexible base member; and
a non-perforated section of the flexible base member carrying a scented portion.

2. The skin patch of claim 1, wherein the scented portion includes menthol, camphor, and/or eucalyptus.

3. The skin patch of claim 1, wherein the non-perforated section is disc-shaped and is positioned at a center of the flexible base member.

4. The skin patch of claim 1, wherein the perforations are circular.

5. The skin patch of claim 1, wherein the perforations are evenly distributed across the flexible base member.

6. The skin patch of claim 1, wherein the scented portion includes a liquid absorbent material.

7. The skin patch of claim 1, wherein the outer perimeter defines a trapezoid with the upper edge being longer than the lower edge.

8. The skin patch of claim 1, wherein the flexible base member carries the scented portion on the first side.

9. The skin patch of claim 1, wherein the flexible base member carries the scented portion within the flexible base member.

10. The skin patch of claim 1, wherein the scented portion remains on the flexible base member when nasal drainage is wiped from the flexible base member.

11. A method comprising delivering scent to a human nose, by adhering a skin patch to an upper lip, the skin patch including:
a flexible base member configured to be adhered between a base of the nose and a vermillion border; the flexible base member defining an outer perimeter having a left edge, a right edge, an upper edge extending from the left edge to the right edge, and a lower edge opposite the upper edge and extending from the left edge to the right edge; the flexible base member including first side and a second side opposite the first side, the second side including a skin contact adhesive;
a plurality of perforations through the flexible base member extending from the first side to the second side, the perforations being distributed across the flexible base member; and
a non-perforated section of the flexible base member carrying a scented portion.

12. The method of claim 11, wherein the scented portion includes menthol, camphor, and/or eucalyptus.

13. The method of claim 11, wherein the non-perforated section is disc-shaped and is positioned at a center of the flexible base member.

14. The method of claim 11, wherein the perforations are circular.

15. The method of claim 11, wherein the perforations are evenly distributed across the flexible base member.

16. The method of claim 11, wherein the scented portion includes a liquid absorbent material.

17. The method of claim 11, wherein the outer perimeter defines a trapezoid with the upper edge being longer than the lower edge.

18. The method of claim 11, wherein the flexible base member carries the scented portion on the first side.

19. The method of claim 11, wherein the flexible base member carries the scented portion within the flexible base member.

20. The method of claim 11, wherein the scented portion remains on the flexible base member when nasal drainage is wiped from the flexible base member.
